(19) United States
(12) Patent Application Publication
(10) Pub. No.: US 2005/0059129 A1
(43) Pub. Date: Mar. 17, 2005

(54) Biologically active material conjugated with biocompatible polymer with 1:1 complex, preparation method thereof and pharmaceutical composition comprising the same

(76) Inventor: Myung-Ok Park, Seoul (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/947,513

(22) Filed: Sep. 22, 2004

Related U.S. Application Data
(63) Continuation-in-part of application No. PCT/KR04/00701, filed on Mar. 27, 2004.

(30) Foreign Application Priority Data
Mar. 28, 2003 (KR) 10-2003-0019734
Jun. 2, 2004 (KR) 10-2004-0007983

(51) Int. Cl. C08G 63/48; C08G 63/91; C12N 9/02; C12N 9/16; C12N 9/20; C12N 9/24

(52) U.S. Cl. 435/189; 435/200; 530/351; 530/388.1; 530/383; 530/399; 435/198; 435/196; 525/54.1

(57) ABSTRACT

The present invention relates to conjugates of biocompatible polymers and biologically active molecules wherein the activated biocompatible polymer is conjugated to a carboxyl group of biologically active material at a molar ratio of 1:1 and methods of preparation thereof and a pharmaceutical composition comprising the same.

### RELATED APPLICATIONS

This application is a continuation-in-part of International Application No. PCT/KR2004/000701, filed Mar. 27, 2004 which designates the United States and claims priority to Korean Patent Application No. 10-2004-0007983, filed Feb. 6, 2004 and Korean Patent Application No. 10-2003-0019734, filed Mar. 28, 2003. The International Application has not yet been published but is expected to be published in a language other than English.

### FIELD OF THE INVENTION

The present invention relates to conjugates of biocompatible polymers and biologically active molecules with a molar ratio of 1:1 and methods of preparation thereof and a pharmaceutical composition comprising the same. Particularly, the present invention relates to conjugates formed by specifically binding biocompatible polymers to a carboxyl group of biologically active molecules at a molar ratio of 1:1 and methods of preparation thereof, and a pharmaceutical composition comprising the same.

### BACKGROUND OF THE INVENTION

Use of proteins and peptides as medicinal products generally has been limited by several problems. For example, peptides or proteins are very low in in vivo absorption efficiency because they are easily hydrolyzed or degraded by proteases within a short period of time after being taken into the body, and also induce immune response with repeated administration. Therefore, most protein and peptide drugs have been required to be administered by injection at least once a day or more. This frequent administration by injection, however, causes pain and risk to patients. Also, frequent injections over long periods is costly and inconvenient to the patients.

Attempts to develop more stable drugs are required to solve the above problems, and the technology to modify biologically active materials such as proteins or polypeptides with biocompatible polymers has been developed. Conjugates of proteins or pharmaceutically active molecules to biocompatible polymers can afford great advantages when they are applied in vivo and in vitro. When being covalently bonded to biocompatible polymers, biologically active materials can exhibit modified surface properties and solubility, and thus can be increased in solubility within water or organic solvents. Further, the presence of biocompatible polymers can make the proteins and/or polypeptides conjugated to them more stable in vivo, increase biocompatibility of the proteins and reduce immune response, and reduce the clearance rate of the proteins by the intestine, the kidney, the spleen, or the liver.

Although the conjugation of the biologically active materials such as a protein or peptide of interest with biocompatible polymers such as PEG has many advantages, some problems remain in conjugating by known methods.

The most common conjugation method is achieved by bonding activated PEG to the amino group of amino acid residues such as lysine. However, because one or more free lysine residues in many proteins are frequently located at or adjacent to the active site, when a lysine residue is used for the conjugation, it tends to decrease the biological activity of the PEG-protein conjugates substantially. Furthermore, because the lysine residue of proteins reacts easily with PEG, PEG-protein conjugates with two or more PEG molecules attached to one protein molecule may be obtained. For example, when more than two PEG molecules bind to the surface of cytokines such as interferon, CSF, and interleukin or polypeptides such as EGF, hGH, and insulin, the biological activity of conjugate is rapidly reduced resulting in loss of function. Additionally, since these reactions tend to occur randomly, a mixture of many kinds of PEG-protein conjugates is produced, which makes the purification of the desired conjugates complicated and difficult. If too many polymer molecules are attached to targeting proteins or peptides, the conjugates lose all or much of their biological activity. Also, if an expressively reactive linker has been used or insufficient numbers of polymers are attached to targeting protein molecules, the therapeutic efficacy of those conjugates can be decreased.

To overcome these problems, many attempts have been made to conjugate biocompatible polymers to amino acid residues of proteins substituted by genetic engineering to conjugate polymers to a specific site of proteins. However, this method generally alters the original properties of proteins. Also the safety of these genetically engineered molecules as therapeutic drugs needs to be proved.

Attempts have been made to solve the problems by chemically modifying specific sites of biologically active materials with biocompatible polymers. U.S. Pat. Nos. 5,951,974 and 5,985,263 describes conjugation of PEG molecules to the histidine residue of interferon to increase the efficacy of drugs by lengthening half-life in vivo and the like. However, this method still used the reactive amino group and produced isomers of PEG-IFN randomly attached at several histidine sites, and requires an additional purification step using an ion-exchange column to separate the desired 1:1 complex of highly active PEG-IFN conjugate. Further, the imidazolyl group of histidine to which PEG is attached is easily hydrolyzed compared to other amino groups of amino acids, and interferon is easily released from the PEG-interferon conjugate.

U.S. Pat. No. 5,766,897 describes conjugation of macromolecules and mutant forms thereof at their cysteine residues to activated PEG molecules. Because of disulfide bond most protein molecules have either one free or no spare cysteine. Thus, an amino acid which is not related to the active site can be changed to a cysteine residue by mutagenesis to conjugate the new cysteine residue with polymers. This method, however, tends to produce conjugates with significantly decreased activity compared to conjugates at amino or carboxyl groups of proteins, although it has an advantage of attaching the polymer to a specific site of biologically active molecules.

U.S. Pat. No. 5,985,265 describes site-specific conjugates at N-terminal residues of G-CSF and IFN with PEG molecules. However, reactivity of these activated polymers is low, and the reaction needs a longer reaction time. In addition, the yield of the reaction is low and stability of proteins is poor. In case that the active site of protein molecules is especially near the N-terminus, conjugation at the N-terminal amino group results in the significant decrease or loss of biological activity.

U.S. Pat. No. 5,824,778 describes conjugates of G-CSF at amino and carboxyl groups by PEG. Excess EDAC was added to activate the carboxyl groups of the protein and many PEG molecules were attached to activated carboxyl groups of several residues. The obtained PEG-G-CSF conjugate has been determined to be a heterogeneous mixture having various numbers of PEG molecules attached, and biological activity of the conjugate was significantly reduced. Therefore, if the biological activity of biologically active molecules can be maintained after conjugation with the polymer at a desired ratio, and a homogenous species of site-specific conjugates can be obtained, clinical usefulness of such molecules will increase remarkably.

### SUMMARY OF INVENTION

Inventors of present invention prepared PEG-biologically active molecule conjugates at a ratio of 1:1, wherein PEG is attached to a carboxyl group of biologically active molecules. Carboxyl groups of biologically active molecules have lower reactivity than amino groups. It was observed that these conjugates show therapeutic efficacy up to 20-fold higher than native(non-conjugated) proteins because they have an extended half-life and higher stability compared to native proteins. Also they observed that the 1:1 complex showed superior characteristics to conjugates of higher than 1:1 molar ratio at carboxyl groups or conjugates at amino groups.

Therefore, the present invention provides conjugates of biologically active molecules with biocompatible polymers wherein biocompatible polymers are specifically attached to a carboxyl group of biologically active molecules at a ratio of 1:1, methods of preparation thereof and a pharmaceutical composition comprising the same. The conjugates of the present invention retain biological activity of native biologically active molecules and have increased stability, bioavailability, and half-life.

### BRIEF DESCRIPTION OF THE DRAWINGS

FIG. 1 shows the degree of conjugation for mPEG(5K)-Hz-G-CSF by HPLC (FIG. 1A) and SDS-PAGE (FIG. 1B).

FIG. 2 shows the degree of conjugation for mPEG(20k)-Hz-G-CSF by HPLC (FIG. 2A) and SDS-PAGE (FIG. 2B).

FIG. 3 shows mPEG(5K)-Hz-IFN with the molar ratio of 1:1 on SDS-PAGE.

FIG. 4 shows the productivity of mPEG(20k)-Hz-IFN conjugate according to the amount of EDAC added on SDS-PAGE.

FIG. 5 shows the degree of reactivity for mPEG(20k)-Hz-IFN conjugate according to the addition method of EDAC and the amount of EDAC on SDS-PAGE.

FIG. 6 shows SDS-PAGE of mPEG(20k)-Hz-IFN conjugate purified by an ion-exchange column.

FIG. 7 shows the comparison of the biological activity of mPEG(20k)-Hz-G-CSF conjugate, native G-CSF, and Neulasta™ (PEG-G-CSF, developed by Amgen, FDA approved in 2002) by cell based assay.

FIG. 8 shows the plasma half-life of mPEG(20k)-Hz-G-CSF, native G-CSF, and Neulasta™(PEG-G-CSF, developed by Amgen, FDA approved in 2002).

FIG. 9 shows WBC of mPEG(20k)-Hz-G-CSF conjugates, native G-CSF, and Neulasta™(PEG-G-CSF, Developed by Amgen, FDA approved in 2002).

FIG. 10 shows biological activity of mPEG(12K)-Hz-IFN conjugate, native IFN, and PEG-IFN(developed by Schering-Plough) by CPE assay.

FIG. 11 shows the comparison of biological activity of mPEG(20k)-Hz-IFN conjugate with native IFN by CPE assay.

FIG. 12 shows the comparison of biological activity between Di-mPEG-Hz-IFN, two PEG molecules attached to one IFN molecule and mono-mPEG-Hz-IFN, one PEG molecule attached to one IFN molecule, by CPE assay.

FIG. 13 shows the comparison of half-life in plasma of PEG(20k)-Hz-IFN conjugate, native IFN, and PEG-IFN(developed by Schering-Plough).

FIG. 14 shows the comparison of stability between PEG-IFN conjugated at a carboxyl group (FIG. 14B) and at an amino group (FIG. 14A).

FIG. 15 shows the HPLC chromatogram for native PTH, which has not been modified by biocompatible polymers.

FIG. 16 shows the HPLC chromatogram for a reaction mixture(unreacted PTH, mPEG(20k)-Hz-PTH, mPEG(20k)-Hz) of PTH with mPEG(20k)-Hz before purification (peak 1: unreacted PTH with PEG polymer, peak 2: mPEG(20k)-Hz-PTH)

FIG. 17 shows HPLC chromatogram of the purified mPEG(20k0)-Hz-PTH after conjugating PTH with mPEG(20k)-Hz.

FIG. 18 shows SDS-PAGE, stained with Coomassie blue for the reaction product between PTH and mPEG(20k)-Hz (lane 1: MW marker, lane 2: PTH, lane 3: PTH, PTH-mPEG(20k)-Hz conjugate before purification, lane 4: PTH-mPEG(20k)-Hz conjugate after purification.

FIG. 19 shows the in vivo biological activity for PTH and PEG-PTH conjugate.

FIG. 20 shows the half-life of PTH and PEG-PTH conjugates in rats.

FIG. 21 shows the HPLC chromatogram for purified PEG-EPO with 1:1 complex.

FIG. 22 shows a cell proliferation assay of native EPO and PEG-EPO.

### DETAILED DESCRIPTION OF THE INVENTION

In an aspect, the present invention relates to the conjugates of biocompatible polymer-biologically active material, wherein the activated biocompatible polymer is conjugated to a carboxyl group of biologically active material at a molar ratio of 1:1.

In another aspect, the present invention relates to a pharmaceutical composition comprising a therapeutically effective amount of the above biocompatible polymer-biologically active molecule conjugates and pharmaceutically acceptable carriers.

In a further aspect, the present invention relates to a method of preparation of conjugates of biocompatible polymer-biologically active material at a molar ratio of 1:1, wherein the biocompatible polymer is conjugated at a carboxyl group of biologically active material, comprising the step of conjugating the biologically active material to the activated biocompatible polymer with the stepwise addition of coupling reagent under the condition wherein the molar ratio of biologically active material to activated the biocompatible polymer is 1:1 to 1:20, the ratio of biologically active material to the coupling reagent is 1:1 to 1:50, and pH is in the range of 2 to 5.

In the above method, EDAC, as an example of the coupling reagent, was added stepwise more than 5 times, preferably 5 or 6 times, because EDAC is readily hydrolyzed in aqueous solution.

The method described above provides the conjugates wherein biocompatible polymers are attached to a carboxyl group of biologically active molecules at a ratio of 1:1. In other words, the present invention provides site specific conjugation by binding activated polymers to a carboxyl group of biologically active materials at a molar ratio of 1:1. These conjugates retain the biological activity of biologically active materials by preventing the attachment of polymers to active sites. The present invention also provides the conjugates with a molar ratio of 1:1 by avoiding the random reaction with many reactive residues at active sites to produce various kinds of heterogeneous mixtures. Further, the conjugates of the present invention have several advantages such as increased stability in vivo, increase of bioavailability, and extended half-life caused by biocompatible polymers. Therefore, production of homogeneous biocompatible polymers-biologically active material conjugates of the present invention provides a cost and time effective process, as compared to other processes of the prior art.

WO92/16555 describes the reaction of ovalbumin at the carboxyl or carbohydrate group with PEG-hydrazide containing amino acid spacer. However, it only describes the conjugates with a number of PEG molecules attached without mentioning a method of preparation for conjugates of biologically active materials with biocompatible polymers with the ratio of 1:1, and the biological activity of conjugates.

Also, U.S. Pat. No. 5,824,779 describes linkage of PEG to the carboxyl group of G-CSF but the conjugate prepared according to their method had very low activity because several PEG molecules were randomly attached to aspartic acids or glutamic acids of G-CSF.

There have been problems to control reaction condition for specific linkage or the number of attaching polymers using the difference in reactivity according to pKa of amino acids.

When an attempt was made to react the amino group of lysine in the range of pH 7 to 8, the histidine groups also reacted randomly. And, when pH was lowered to about 6 to 6.5, histidine groups became more reactive with PEG than lysine groups(U.S. Pat. No. 5,951,974 and U.S. Pat. No. 5,985,263). Inventors of the present invention observed that PEG can be attached to carboxyl groups, especially C-terminus of biologically active materials at a ratio of 1:1 at pH equal to or lower than 3 according to the above method.

Therefore in one aspect, the present invention relates to the conjugate of biocompatible polymer-biologically active material, wherein the biocompatible polymer is conjugated to the C-terminus of the biologically active material at a molar ratio of 1:1.

In another aspect, the present invention relates to a pharmaceutical composition comprising a pharmaceutically acceptable amount of the conjugate, wherein the biocompatible polymer is conjugated to the C-terminus of the biologically active material at a molar ratio of 1:1 and pharmaceutically acceptable carriers.

In another further aspect, the present invention relates to a method of preparation of conjugate of biocompatible polymer-biologically active material at the C-terminus of the biologically active material with a molar ratio of 1:1, comprising the step of conjugating the biologically active material to the activated biocompatible polymer with the stepwise addition of coupling reagent under the condition wherein the molar ratio of biologically active material to the activated biocompatible polymer is 1:1 to 1:20, the ratio of biologically active material to the coupling reagent is 1:1 to 1:50, and pH is in the range of 2 to 5.

Biocompatible Polymers

The term “conjugating material” used for conjugation of biologically active molecules means any biocompatible polymer which can be linked to biologically active molecules such as natural or synthetic polymers.

The term “biocompatibility” means biocompatible with living tissues or systems, and being nontoxic, noninflammatory, and noncarcinogenic without causing harm, inflammation, immune response and carcinogenesis in the body.

Biocompatible polymers are conjugated with biologically active materials. The useful polymers of the present invention are readily soluble in various solvents and have molecular weight of between about 300 and about 100,000 Da and preferably between about 2,000 and about 40,000 Da. The biocompatible polymers include, but are not limited to, polyethylene glycol (PEG), polypropylene glycol (PPG), polyoxyethylene (POE), polytrimethylene glycol, polylactic acid and its derivatives, polyacrylic acid and its derivatives, polyamino acid, polyvinylalcohol, polyurethane, polyphosphazene, poly(L-lysine), polyalkylene oxide (PAO), polysaccharide, dextran, polyvinyl pyrrolidone, polyacrylamide, copolymers thereof and other nonimmunogenic polymers.

Biocompatible polymers of the present invention are intended to include not only linear polymers but also polymers as follows. Biocompatible polymers of the present invention include soluble, non-antigenic polymers linked to an activated functional group that is capable of being nucleophilically substituted through an aliphatic linker residue (U.S. Pat. Nos. 5,643,575 and 5,919,455). Also, biocompatible polymers of the present invention include multi-armed, mono-functional and hydrolytically stable polymers, having two linker fragments which have polymer arms around a central carbon atom, a residue which is capable of being activated for attachment to biologically active materials such as proteins, and side chains which can be hydrogen or methyl group, or other linker fragment(U.S. Pat. No. 5,932,462). In addition, biocompatible polymers of the present invention include polymers of branched PEG in which the functional groups of polymers are attached to biologically active materials via linker arms having reporter residues(WO 00/33881).

Among them, PEG is one of the most common biocompatible polymers of the present invention. In general, PEG is a nontoxic hydrophilic polymer having the repeating unit, HO—(CH2CH2O)n—H. Various proteins are reported to show extended half-lives, increased solubility, increased stability, and reduced immunogenicity in plasma when being conjugated with PEG.

The range of molecular weight of PEG molecules conjugated to biologically active materials such as proteins or peptides is from about 1,000 to 100,000 Da and the toxicity of PEG over 1,000 Da is known to be very low. PEGs in the range of from 1,000 to 6,000 Da are distributed to the whole body and cleared in the kidney. Branched PEG with molecular weight of 40,000 Da are distributed in blood or organs including the liver, and metabolized in the liver.

PEG is the most preferable biocompatible polymer because PEG is commercially available in the various molecular weight ranges, each oxyethylene unit is hydrophilic to be accessible to bind 2-3 water molecules, PEG derivatives with one-terminal functional group from methoxy polyethylene glycol are easy to synthesize, PEG has very low risk of antigen-antibody reaction, and the related technology is well developed.

Biologically Active Materials

The term “biologically active molecule” means all nucleophiles conjugated with activated biocompatible polymers, and which retain at least some of their biological activity after conjugation. The term “biologically activity” used herein is not limited by physiological or pharmacological activity. For example, some conjugates of nucleophilic containing enzymes can catalyze reactions in organic solvents. Similarly, some polymer conjugates including proteins such as Con-canavalin A, or immunoglobulin can also be used in diagnostics in the laboratory. In general, biologically active molecules can be isolated from nature or synthesized recombinantly or chemically, and include proteins, peptides, polypeptides, enzymes, biomedicines, genes, plasmids, or organic residues.

Proteins, peptides, and polypeptides of interest include, but are not limited to, hemoglobin, serum proteins(for example, blood factors including Factor VII, VIII, and IX), immunoglobulins, cytokines(for example, interleukins), α-, β- and γ-interferons, colony stimulating factors including G-CSF and GM-CSF, platelet derived growth factor(PDGF), phospholipase-activating protein(PLAP), and parathyroid hormone(PTH). Other proteins of general biological or therapeutic interest include insulin, plant proteins (for example, lectins and ricins), tumor necrosis factors(TNF) and related alleles, growth factors(for example, tissue growth factors such as TGFα and TGFβ and epidermal growth factors), hormones (for example, follicle-stimulating hormone, thyroid-stimulating hormone, antidiuretic hormones, pigmentary hormones, luteal hormone-releasing hormone and derivatives thereof), calcitonin, calcitonin gene related peptide(CGRP), synthetic enkephalin, somatomedins, erythropoietin, hypothalamic releasing factors, prolactin, chorionic gonadotropin, tissue plasminogen activator, growth hormone releasing peptide(GHRP), thymic humoral factor(THF) and the like. Immunoglobulins of interest include IgG, IgE, IgM, IgA, IgD, and fragments thereof.

When two or more biocompatible polymers are attached to especially low molecular weight polypeptides such as Interferon and G-CSF, these conjugates exhibit considerably low biological activity. Also, two or more polymers are attached when relatively highly reactive amino groups are conjugated and thus the separation of conjugates in a 1:1 complex is not easy. However, the present invention provides the selective preparation of conjugates of biocompatible polymer-IFN or biocompatible polymer-G-CSF in a 1:1 complex, wherein these conjugates show high biological activity, increased half-life, and excellent bioavailability.

The biologically active materials of the present invention also include any portion of a polypeptide demonstrating in vivo bioactivity. This includes amino acid sequences, antisense oligomers, antibody fragments, linear antigen(Ref. U.S. Pat. No. 4,946,778), binding molecules including fusions of antibodies or fragments, polyclonal antibodies, monoclonal antibodies, catalytic antibodies, nucleotides, oligonucleotides.

The biologically active materials also include enzymes. Enzymes of interest include carbohydrate-specific enzymes, proteolytic enzymes, oxidoreductases, transferases, hydrolases, lyases, isomerases and ligases. Without being limited to particular enzymes, examples of enzymes of interest include asparaginase, arginase, arginine deaminase, adenosine deaminase, superoxide dismutase, endotoxinases, catalases, chymotrypsin, lipases, uricases, adenosine diphosphatase, tyrosinases and bilirubin oxidase. Carbohydrate-specific enzymes of interest include glucose oxidases, glucosidases, galactosidases, glucocerebrosidases, glucouronidases, etc.

Examples described above are examples of suitable biologically active nucleophiles conjugated with activated biocompatible polymers of the present invention. All suitable biologically active materials with nucleophilic group are to be also included in the present invention although they are not mentioned above. The biologically active materials for the present invention need to possess at least one free carboxyl group for conjugation by polymer.

The conjugates of the present invention are biologically active for the purpose of therapeutic application. Mammals can be treated by administering the therapeutically effective dose of polymer conjugates containing biologically active materials.

Preparation of Biocompatible Polymer-Biologically Active Material Conjugates

To conjugate biocompatible polymers to the biologically active molecules, one of the end groups of polymers is converted into a reactive functional group. This process is referred to as “activation” and the product is called an “activated” polymer. For instance, to conjugate poly(alkylene oxides, PAO) to peptides or proteins, one of the hydroxyl end groups of the polymer can be converted into a reactive functional group such as carbonate and activated PAO is produced, which is soluble at room temperature. This group includes mono substituted poly(alkylene oxide) derivatives such as mPEG or other suitable alkyl-substitute PAO derivatives containing C1-4 end group.

The term “reactive functional group” used in the art and herein is the group or the residue activating biocompatible polymers to bind with biologically active materials.

The reactive functional group of the present invention is selected from the functional groups able to react with carboxylic acid and reactive carbonyl group, for example, primary amine, or hydrazine and hydrazide functional groups (such as acyl hydrazide, carbazate, semicarbazate, thiocarbazate etc.).

The term “coupling reagent of carboxyl group”(hereinafter referred to as coupling reagent) used in the art and herein means any reagent to couple the carboxyl groups of biologically active materials to biocompatible polymers which have been activated at the above reactive functional group.

The coupling reagents of the carboxyl group in the present invention of interest include, but are not limited to, carbodiimidyl coupling agents, for example, EDAC[N-(3-dimethyl-aminopropyl)-N′-ethylcarbodiimide hydrochloride], DIC[1,3-diisopropyl carbodiimide], DCC[dicyclohexyl carbodiimide], and EDC[1-ethyl-3-(3-dimethylamino propyl)-carbodiimide]. The preferable coupling agent for the carboxyl group is EDAC.

The method of preparing the conjugates of the present invention includes the step of reacting biologically active molecules containing nucleophiles capable of performing the substitution reaction with activated biocompatible polymers under the condition in which sufficient conjugation can be possible while retaining at least a portion of intrinsic bioactivity of biologically active molecules.

The biologically active material-biocompatible polymer conjugates with a ratio of 1:1 are obtained by reacting the biologically active materials with a stoichiometric excess amount of polymers. For example, in the preparation of protein-polymer, peptide-polymer, enzyme-polymer, antibody-polymer, and drug-polymer conjugates, the molar ratio of biologically active material to biocompatible polymer is in the range of from about 1:1 to 1:20, more preferably from 1:1 to 1:10. The reagents to activate carboxyl groups of biologically active materials are selected from the group as follows, but are not limited to them. For example, N-(3-dimethyl-aminopropyl)-N′-ethylcarbodiimide hydrochloride(EDAC), water soluble carbodiimide group such as 3-[2-morpholinyl-(4)-ethyl], and 5-substituted isoxazolinium salts such as p-toluene sulfonate, Woodward's Reagent K.

The molar ratio of biologically active materials to EDAC used in the present invention is in the range of from about 1:1 to 1:50, more preferably from about 1:1 to 1:30, and most preferably from about 1:1 to 1:20. However, increased yield of PEG-biologically active material conjugates was observed when the addition of EDAC was divided to more than 5 times, preferably 5 or 6 times rather than adding 20-fold molar excess of EDAC at once because EDAC is readily hydrolyzed in aqueous solution.

The conjugation reaction of biologically active materials with activated polymers is dependent on the pH of water soluble solvents functioning as a buffer. In general, the pH of reaction buffer for proteins/polypeptides is in the range from 2 to 5, preferably from 2.5 to 4.5. The optimum reaction condition for stabilization of these substances and reaction yield has been known in the art. The suitable temperature for the conjugation reaction is in the range of 0 to 60° C. and preferably in the range of 4 to 30° C. The temperature of the solvents should not exceed the denaturation temperature of proteins or peptides. Also, the reaction time of 10 minutes to 5 hours is preferable in this preparation. The conjugates prepared can be recovered and purified by column chromatography, diafiltration or a combination of above two processes.

Pharmaceutical Composition

The present invention also relates to a pharmaceutical composition comprising a therapeutically effective dose of the activated biocompatible polymer-biologically active material conjugates as an active ingredient.

The term “pharmaceutically acceptable” used in the art and herein means not causing allergic reaction or similar reaction when administered to humans.

The biocompatible polymer-biologically active material conjugate as an active ingredient of the pharmaceutical composition can be used itself or formulated in combination with pharmaceutically acceptable carriers for disease prevention and treatment.

The term “pharmaceutically acceptable carrier” used in the art and herein means pharmaceutically acceptable molecules, composition, or vehicles such as solutions, diluents, excipients, or solvents to carry the biologically active materials from one organ or tissues to other organs or tissues. The pharmaceutical composition of the present invention can be administered by the route of oral, local, injection or parenteral route and its formulation include therapeutically effective doses of the biocompatible polymer-biologically active material conjugates as an active ingredient. The formulation for oral administration of the present invention include pills, tablets, coated tablets, granules, troches, wafers, elixirs, hard and soft gelatin capsules, solutions, syrups, emulsions, suspensions, or sprays etc. and for parenteral administration, injectable solutions, microcapsules, patches, and others are included.

The pharmaceutical formulation can be prepared according to the known method by using pharmaceutically acceptable inactive inorganic or organic additives. For example, lactose, corn starch and its derivatives, talc, or stearic acid and its salts can be used to prepare pills, tablets, and hard gelatin capsules. The additives of soft gelatin capsules and suppositories are for example, oil, wax, semi-solid or liquid polyol, and natural or solidified oil. The suitable additives for preparation of solution or syrup are for example, water, sucrose, invertase, glucose, and polyol. The suitable additives for preparation of injectable solution are water, alcohol, glycerol, polyol, plant oil etc. The injectable solution can be used as the combination of preservatives, indolent agents, solubilizers, and stabilizers. The formulation for local administration can be also used as the combination of gas, diluents, lubricants, and preservatives. The suitable additives for microcapsules or transplantation are copolymer or glycolic acid and lactic acid.

The dose of the biocompatible polymer-biologically active material conjugates of the present invention varies depending on the absorption rate of the biologically active materials, solubility, patient's age, sex, condition and severity of diseases, etc. as well known in the art.

Particularly, the administration of biocompatible polymer-biologically active material conjugates of the present invention reduces the injection intervals from daily or once per two days to weekly or biweekly injection. Therefore, the toxicity and site effects of drugs by frequent administration are reduced substantially.

The following examples further describe and demonstrate embodiments within the scope of the present invention. The examples are given solely for the purpose of illustration and are not to intended limit the present invention, as many variations thereof are possible without departing from the spirit and scope of the invention.

### EXAMPLES

1. Preparation of Biocompatible Polymer-Biologically Active Material Conjugates Via a Carboxyl Group of Biologically Active Material

### Example 1

### Preparation of mPEG(12000)-Hz-G-CSF Conjugate

1 mg of G-CSF solution(0.00005 mmol, Dong-A Pharm. LEUCOSTIM) was dialyzed(Centricon-10, Amicon, USA) against 50 mM MES buffer solution (pH 3.0) to the final concentration of 2 mg/ml. To this protein solution, 6.6 mg of mPEG(12000)-Hz(ISU Chemical, Korea, 0.0005 mmol) was added and followed by 2ul (0.001 mmol, 20-fold molar excess) of EDAC solution prepared by dissolving 2 mg of EDAC in 20 ul of d-H2O. The reaction was carried out for 1 hour at room temperature(20-25° C.) with stirring. After 1 hour, unreacted G-CSF and excess reagent were removed by size exclusion column or ion-exchange column. More than 0.3 mg of mPEG(12000)-Hz-G-CSF conjugate was obtained. By changing the amount of EDAC from 20 to 200-fold molar excess and mPEG(12000)-Hz from 10 to 20-fold molar excess, the reaction was repeated. It was observed that two or more mPEG(12000)-Hz were attached to the carboxyl group of G-CSF when the amount of EDAC is over 50-fold molar excess.

### Example 2

### Preparation of mPEG(5000)-Hz-G-CSF Conjugate

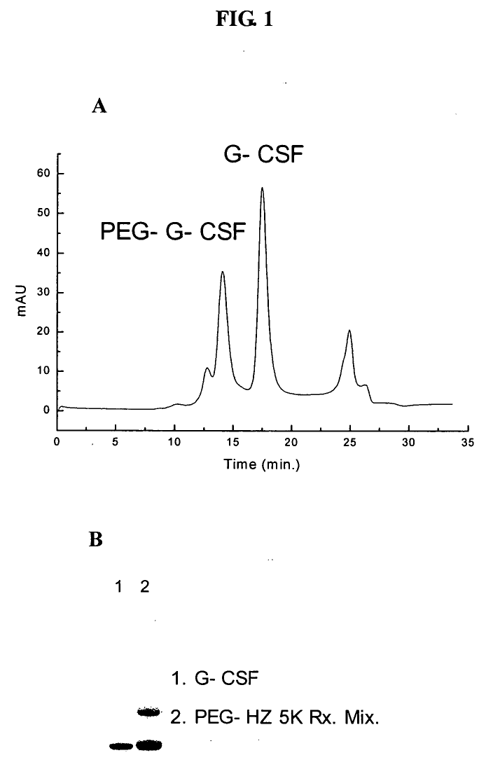

1 mg of G-CSF solution(0.00005 mmol) was dialyzed(Centricon-10, Amicon, USA) against 50 mM MES buffer solution(pH 3.0) to the final concentration of 5 mg/ml. To this protein solution, 1.3 mg of mPEG(5000)-Hz(ISU Chemical, Korea, 0.00025 mmol) was added, followed by 2 ul(0.001 mmol, 20-fold molar excess) of EDAC solution prepared by dissolving 2 mg of EDAC in 20 ul of d-H2O. The reaction was carried out for 1 hour at room temperature(20-25° C.) with stirring. After 1 hour, unreacted G-CSF and excess reagent were removed by size exclusion column or ion-exchange column. More than 0.3 mg of mPEG(5000)-Hz-G-CSF was obtained. FIG. 1 shows the production of mPEG(5000)-Hz-G-CSF conjugate by SDS-PAGE and HPLC profile (size exclusion chromatography).

### Example 3

### Preparation of mPEG(20000)-Hz-G-CSF Conjugate

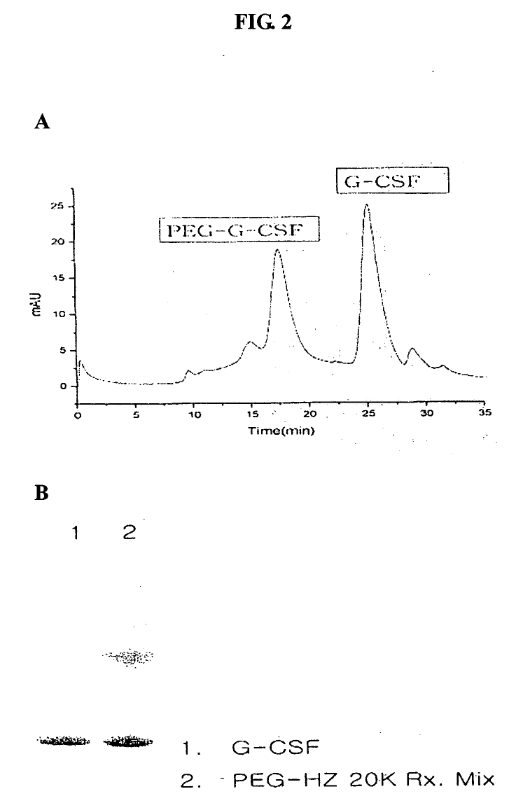

1 mg of G-CSF solution(0.00005 mmol) was dialyzed against 50 mM MES buffer solution(pH 3.0) by ultrafiltration(Centricon-10, Amicon, USA) to the final concentration of 5 mg/ml. To this protein solution, 5 mg of mPEG(20000)-Hz(ISU Chemical, Korea, 0.00025 mmol) was added, followed by 2 ul(0.001 mmol, 20-fold molar excess) of EDAC solution prepared by dissolving 2 mg of EDAC in 20 ul of d-H2O. The reaction was carried out for 1 hour at room temperature(20-25° C.) with stirring. After 1 hour, unreacted G-CSF and excess reagent were removed by size exclusion column or ion-exchange column. More than 0.3 mg of mPEG(20000)-Hz-G-CSF, was obtained. FIG. 2 shows the production of mPEG(20000)-Hz-G-CSF conjugate by SDS-PAGE.

### Example 4

### Preparation of mPEG(5000)-Hz-IFN Conjugate

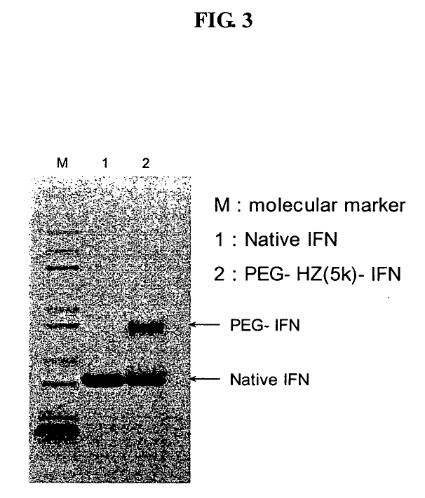

Four tubes each containing 200 ug of IFN solution(0.00001 mmol, Korea Green Cross Corp., Green Alpha) were dialyzed(Centricon-10, Amicon, USA) respectively, against 50 mM MES buffer solution(pH 3.0) to the final concentration of 1 mg/ml. To each tube, 2.16 mg of mPEG(5000)-HZ was added, followed by 0.8 ul(40-fold molar excess) of EDAC solution prepared by dissolving 2 mg of EDAC in 20 ul of d-H2O. The reaction was carried out for 1 hour at room temperature(20-25° C.) with stirring. After 1 hour, unreacted IFN and excess reagent were removed by size exclusion column or ion-exchange column. FIG. 3 shows the conjugation of mPEG(5000)-HZ to IFN molecules by SDS-PAGE.

### Example 5

### Preparation of mPEG(12000)-Hz-IFN Conjugate

1 mg of IFN solution (0.00005 mmol) was dialyzed(Centricon-10, Amicon, USA) against 50 mM MES buffer solution(pH 3.0) to the final concentration of 1 mg/ml. To this protein solution, 6.6 mg of mPEG(12000)-Hz(0.0005 mmol) was added, followed by 2 μl (0.001 mmol, 20-fold molar excess) of EDAC solution prepared by dissolving 2 mg of EDAC in 20 ul of d-H₂O. The reaction was carried out for 1 hour at room temperature(20-25° C.) with stirring. After 1 hour, unreacted IFN and excess reagent were removed by size exclusion column or ion-exchange column. More than 0.3 mg of mPEG(12000)-Hz-IFN was obtained.

### Example 6

### Preparation of mPEG(20000)-Hz-IFN Conjugate

Four tubes containing 200 ug of IFN solution(0.00001 mmol) in each tube were dialyzed(Centricon-10, Amicon, USA) against 50 mM MES buffer solution(pH 3.0) to the final concentration of 2 mg/ml. To each tube, 4.32 mg of mPEG(20000)-Hz(0.0002 mmol, ISU Chemical, Korea) was added, followed by 1 ul(50-fold molar excess) or 4 ul(200-fold molar excess) of EDAC solution prepared by dissolving 2 mg of EDAC in 20 ul of d-H2O. In addition, 30-fold molar excess of sulfo-NHS was added to accelerate the reaction and the results were compared. The reaction was carried out for 1 hour at room temperature(20-25° C.) with stirring. The reaction condition of each sample is described in the table below. After 1 hour, unreacted IFN and excess reagent were removed by size exclusion column or ion-exchange column. The yield of each reaction was compared by SDS-PAGE.

TABLE 1

| Sample No. | IFN | mPEG-Hz | Buffer | Rx. Time |
|---|---|---|---|---|
| #1 | 2 mg/ml | 20 K(×20) | 50 mM MES pH 4.4 | 1 hr |
| | | | EDAC(×50) | |
| #2 | 2 mg/ml | 20 K(×20) | 50 mM MES pH 4.4 | 1 hr |
| | | | EDAC(×50) | |
| | | | NHS(×30) | |
| #3 | 2 mg/ml | 20 K(×20) | 50 mM MES pH 4.4 | 1 hr |
| | | | EDAC(×200) | |

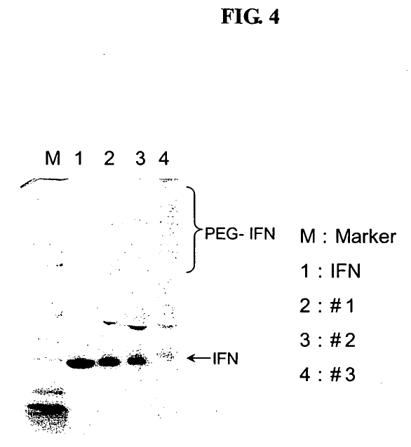

As a result, the reaction with 200-fold molar excess of EDAC showed that too many PEGs were attached to the carboxyl group of IFN and separation of the PEG-IFN conjugate with the molar ratio of 1:1 was not successful and estimation of the number of PEGs attached was not easy. Also, the reaction with 50-fold molar excess of EDAC proceeded but PEG-IFN conjugate with the ratio of 1:1 was not readily distinguishable because the PEG-IFN conjugate was diffused on SDS-PAGE due to the excess amount of EDAC used. When sulfo-NHS was added to the reaction to enhance the efficiency, there was no difference from a control in which sulfo-NHS was not added(FIG. 4).

Also, the reaction efficiency upon adding EDAC several times was performed according to reaction conditions as described in Table 2 below.

TABLE 2

| Sample | | | | Rx. |
|---|---|---|---|---|
| No. | IFN | mPEG-Hz | I. BUFFER | Time |
| #1 | 5 mg/ml | 20 K(×5) | 50 mM MES pH 4.4 | 1 hr |
| | | | EDAC(×10) added 6 times every | |
| | | | 10 min. | |
| | | | Total molar excess of EDAC: 60 | |
| | | | times | |
| #2 | 5 mg/ml | 20 K(×5) | 50 mM MES pH 4.4 | 1 hr |
| | | | EDAC(>10) added 5 times every | |
| | | | 10 min. | |
| | | | Total molar excess of EDAC: 50 | |
| | | | times | |
| #3 | 5 mg/ml | 20 K(×5) | 50 mM MES pH 4.4 | 1 hr |
| | | | EDAC(×5) added 6 times every | |
| | | | 10 min. | |
| | | | Total molar excess of EDAC: 30 | |
| | | | times | |
| #4 | 5 mg/ml | 20 K(×5) | 50 mM MES pH 4.4 | 1 hr |
| | | | EDAC(×3) added 6 times every | |
| | | | 5 min. | |
| | | | Total molar excess of EDAC: 18 | |
| | | | times | |

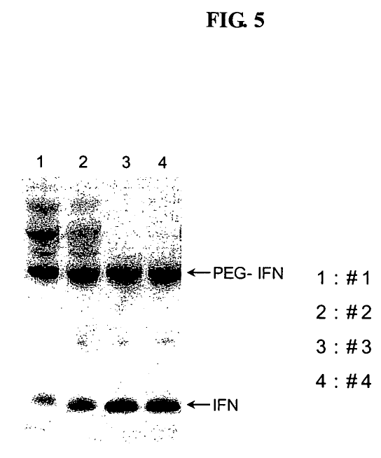

As a result, when EDAC was added stepwise to the reaction mixture, the yield of mPEG-Hz-IFN conjugate with the molar ratio of 1:1 was high(FIG. 5). When the amount of EDAC was over 50-fold molar excess although addition of EDAC was performed stepwise, two or more PEGs were randomly attached to the IFN as shown in FIG. 5.

### Example 7

### Preparation of mPEG(20000)-Hz-IFN Conjugate

1 mg of IFN solution(0.00005 mmol) was dialyzed(Centricon-10(Amicon, USA)) against 50 mM MES buffer solution(pH 2.5) to the final concentration of 5 mg/ml. To this protein solution, 10.8 mg of mPEG(20000)-Hz(0.0005 mmol, 10-fold molar excess) was added, followed by 2 μl (0.001 mmol, 20-fold molar excess) of EDAC solution prepared by dissolving 2 mg of EDAC in 20 ul of d-H2O. The reaction was carried out for 1 hour at room temperature(20-25° C.) with stirring. After 1 hour, unreacted IFN and excess reagent were removed by size exclusion column or ion-exchange column. More than 0.3 mg of mPEG(20000)-Hz-IFN conjugate was obtained.

### Example 8

### Purification of mPEG(20000)-Hz-IFN Conjugates with the Molar Ratio of 1:1

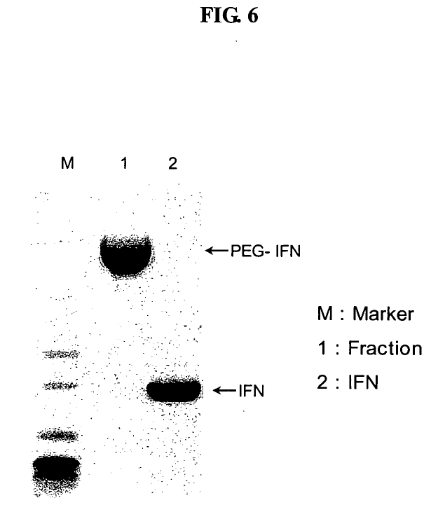

mPEG(20000)-Hz-IFN conjugate(Example 6) was diluted with 10 mM sodium acetate buffer(pH4.4) to the final concentration of 1 mg/ml. mPEG(20000)-Hz-IFN reaction mixture was loaded onto SP-sepharose Fast Flow column(5×50 mm, total 1 ml column vol.), which had been previously equilibrated with 10 mM sodium acetate buffer solution(pH 4.4). After washing the column with 3 column volumes of 10 mM acetate buffer(pH 4.4), a gradient of 10 mM acetate buffer(pH 4.4) containing 500 mM NaCl was used to separate mPEG(20000)-Hz-IFN with the molar ratio of 1:1 from unreacted intact IFN. The above purified mPEG(20000)-Hz-IFN was confirmed to be the conjugate wherein one PEG was attached to the carboxyl group of IFN(FIG. 6).

### Example 9

### Determination of Biological Activity of PEG-G-CSF Conjugate CPE(Cytopathic Effect) Assay was Performed as Follows.

2.5×10⁶ cells(5×10⁵ cells/ml) of M-NFS-60 were sub-cultured on 60 mm dishes(RPMI-1640 media, 10% FBS, 37° C., 5% CO₂). Each of native G-CSF(control) and mPEG(20000)-Hz-G-CSF(Example 3) was diluted to the concentration of 1 ng/μl and added to the 96 well plate containing 1×10⁴ cells in each well, followed by serial dilution. The plate was incubated at 37° C. for 2 days. Then each well was treated with 50 μl of XTT kit(Roche, Germany) and incubated for another 4 hours at 37° C., and O.D. value of the plate was read at 490 nm using ELISA reader.

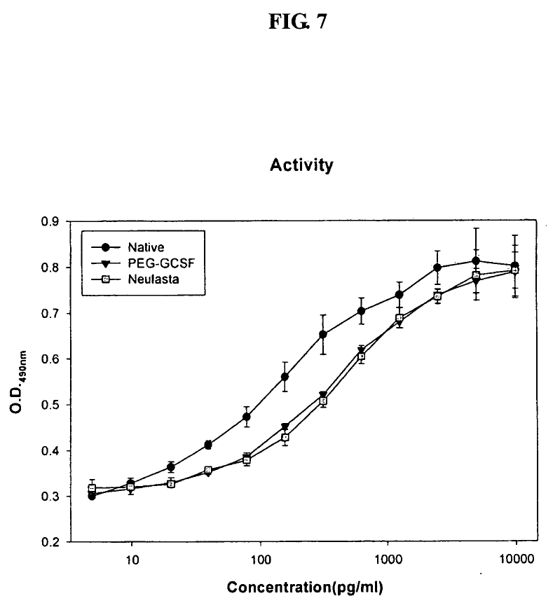

As a result, mPEG(20000)-Hz-G-CSF of the present invention was shown to retain a similar activity to mPEG(20000)-G-CSF conjugate, wherein PEG was attached to the amino group of G-CSF(FIG. 7).

### Example 10

### Determination of Half Life of Peg-G-CSF Conjugate

7-week old Sprague-Dawley rats (5 rats per group) weighing 220-240 g were anesthetized using Ketamin/Rompun and a PE tube was inserted to the vena cava of each rat by surgery. After the rat recovered, 100 ug/kg of mPEG(20000)-Hz-G-CSF(Example 3) was administered through intravenous injection. PBS and 100 ug/kg of native G-CSF were used as placebo and control, respectively, for comparison.

300 ul of blood was withdrawn through the cannula at time intervals of 0, 0.5, 1, 2, 4, 6, 12, 24, and 48 hours after injection. The serum was separated by centrifugation(13,000 rpm, 10 min, 4° C.) and stored at −20° C. for further study.

After incubation the cells for 24 hours with G-CSF free media, each well of 96 well plate was added with 1.5×10⁴ cells. Each serum sample stored as described above was diluted 100 times and 50 ul of the diluted samples was added to each well of 96 well plate. The dishes were incubated at 37° C. for 48 hours under CO₂ gas. Then each well was treated with 50 μl of XTT kit(Roche, Germany). Plates were incubated for another 4 hours at 37° C. and the O.D. value of the plate was read at 490 nm using an ELISA reader.

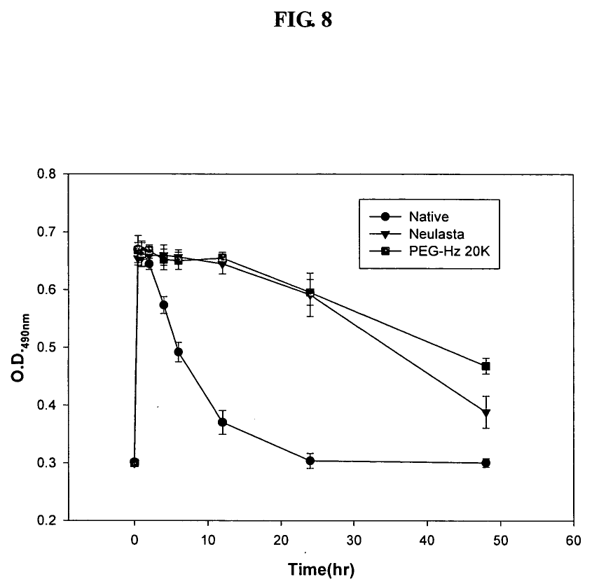

The half-life of mPEG(20000)-Hz-G-CSF(Example 3) is compared to that of native G-CSF and Neulasta™ (PEG attached to N-terminal of G-CSF, Amgen) in FIG. 8. mPEG(20000)-Hz-G-CSF(Example 3) of the present invention showed a much longer half-life compared to native G-CSF, and has similar half-life to that of Neulasta™

### Example 11

### Determination of White Blood Cell (WBC) Count of PEG-G-CSF-Treated Rats

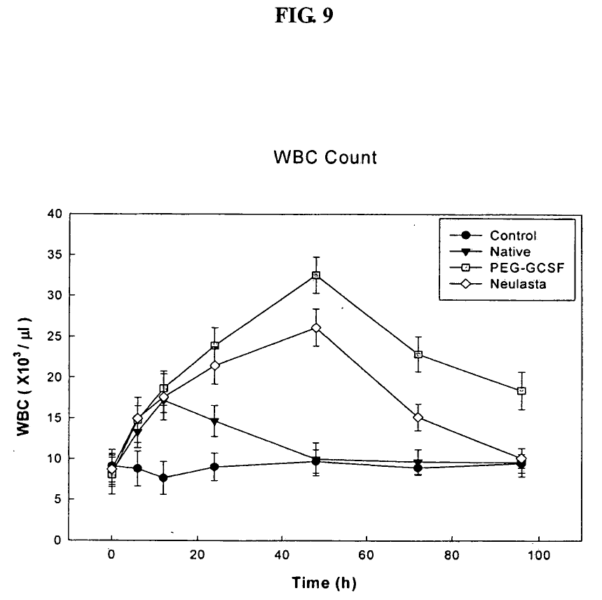

7-week old Sprague-Dawley rats weighing 220-240g were purchased from Charles River Co.(Atsugi, Japan). 100 ug/kg of mPEG(20000)-Hz-G-CSF (Example 3) was injected to the tail vein of rats. The same amount of native G-CSF and saline solution was injected respectively as a control. Blood samples were withdrawn at time intervals of 0, 6, 12, 24, 48, 72, 96 hrs after injection through the tail vein. WBC count was measured by Automated Hematology Analyzer (Cysmex K-4500) as shown in FIG. 9. As a result, mPEG(20000)-Hz-G-CSF of the present invention showed higher WBC counts than both native G-CSF and Neulasta™.

### Example 12

### Determination of Biological Activity of PEG-IFN Conjugate

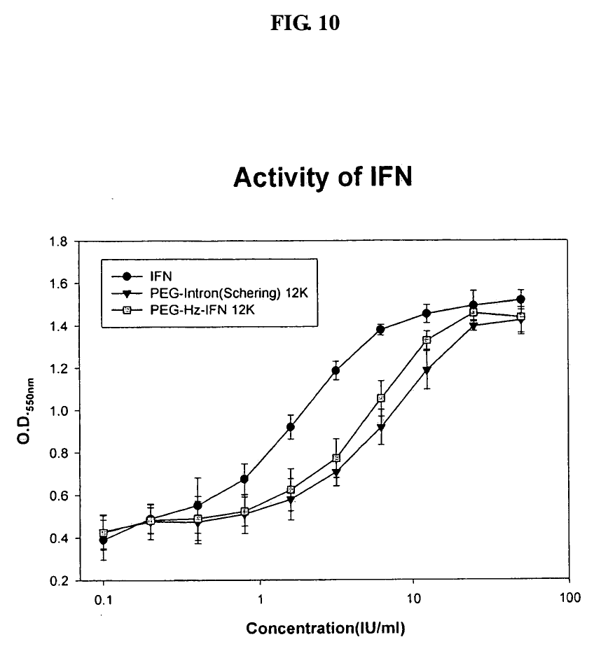

MDBK cells counted in a concentration of 7.5×10⁵ cells/ml using a hemocytometer, were suspended in 5% FBS/MEM media. mPEG(12000)-Hz-IFN(Example 5) was diluted to the concentration of 100 IU(1 mg/ml=2×10⁸ IU). Each well was supplemented with 100 ul of 5% FBS/MEM media and 100 ul of the diluted samples was added to the first well followed by serial dilution. Then 100 ul of cell suspension was added to each well of 96 well plate. The dishes were incubated at 37° C. for 20 hours. 100 ul of Vesicular Stomatitis Virus(VSV, ATCC VR-158) diluted 100 times was added to each well and incubated another 20 hours at 37° C. The growth medium solution containing Vesicular Stomatitis Virus(VSV, ATCC VR-158) of 96 well plate was removed, 50 ul of 0.05% crystal violet dye solution was added to each well, and O.D. of each well was read at 550 nm by ELISA reader to determine the activity of IFN. As a result, the activity of mPEG(12000)-Hz-IFN (Example 5) was found to retain 40-50% of native IFN activity and showed activity similar to that of the comparative PEG-IFN(Schering-Plough, USA, PEG attached to amino group of IFN, approved by FDA)(FIG. 10).

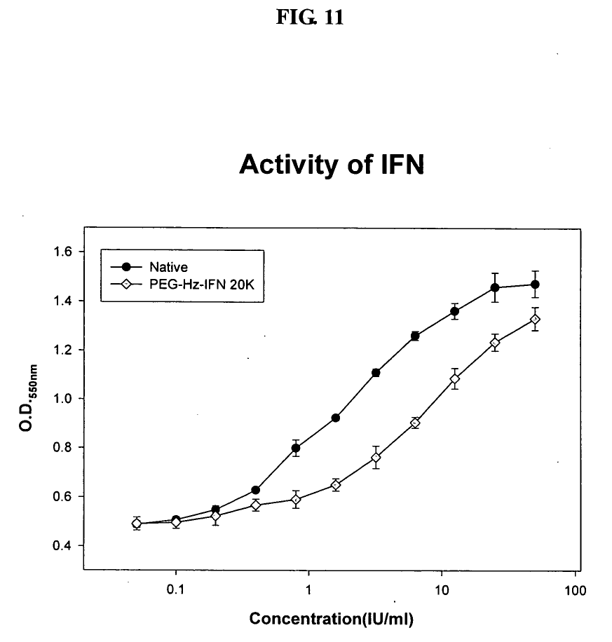

Also, the activity of mPEG(20000)-Hz-IFN (Example 6) was determined to be approximately 40% of native IFN(FIG. 11).

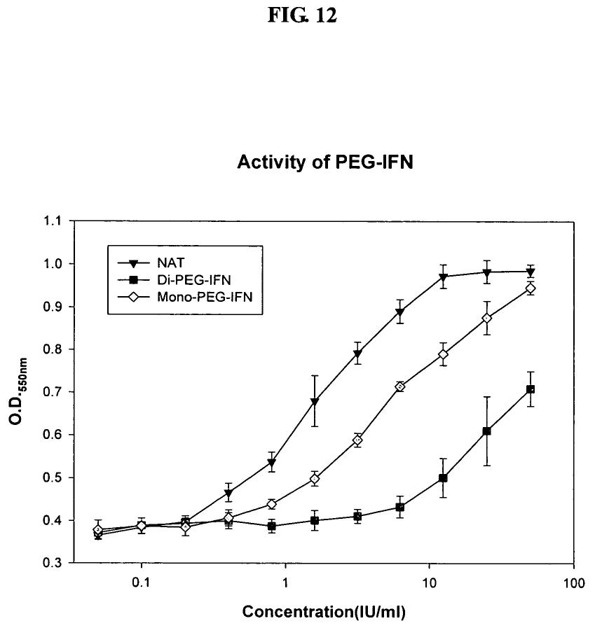

Also, the activity of Di-mPEG-Hz-IFN(Two PEGs attached to one IFN) and mono-PEG-IFN(1:1 complex, one PEG attached to one IFN) was compared by CPE assay and mono-mPEG-Hz-IFN showed high biological activity(FIG. 12).

### Example 13

### Determination of Half-Life of PEG-IFN Conjugate

MDBK cells counted in a concentration of 7.5×10⁵ cells/ml were suspended in 5% FBS/MEM media. 100 ul of cell suspension was put in each well of 96 well plate. Serum was obtained after injecting mPEG(20000)-Hz-INF(Example 6) by intravenous route to rats and diluted 50 times. Each well was added with the diluted serum and incubated in a CO₂ incubator for 20 hours.

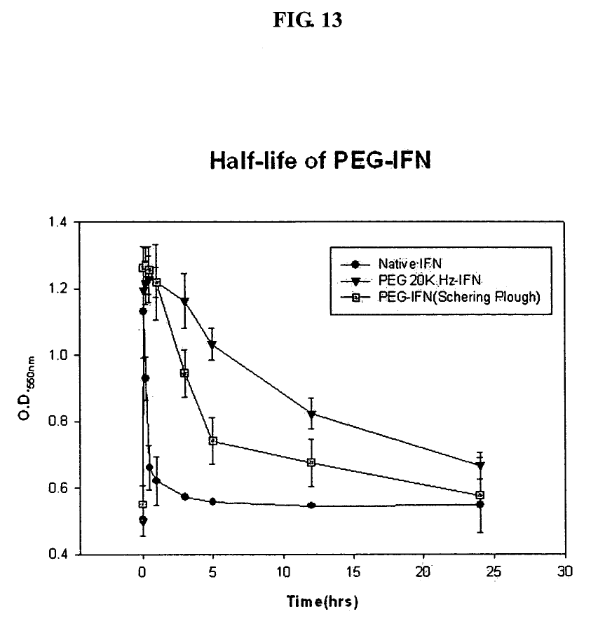

Each well was added with100-fold diluted Vesicular Stomatitis virus(100 ul) and incubated continued for another 20 hours. The virus medium solution in each well was removed, and 50 ul of 0.05% crystal violet dye solution was added to each well. The absorbance at 550 nm was read by a Microplate reader to measure the half life of IFN. FIG. 13 shows the half-life of mPEG(20000)-Hz-IFN(Example 6) conjugated at the carboxyl group and comparison of mPEG(20000)-Hz-IFN with native IFN and comparative product, PEG-IFN. mPEG(20000)-Hz-IFN of the present invention showed a much greater half-life than native IFN and longer half-life than the comparative product, PEG-IFN.

### Example 14

### Stability of PEG-IFN Conjugate

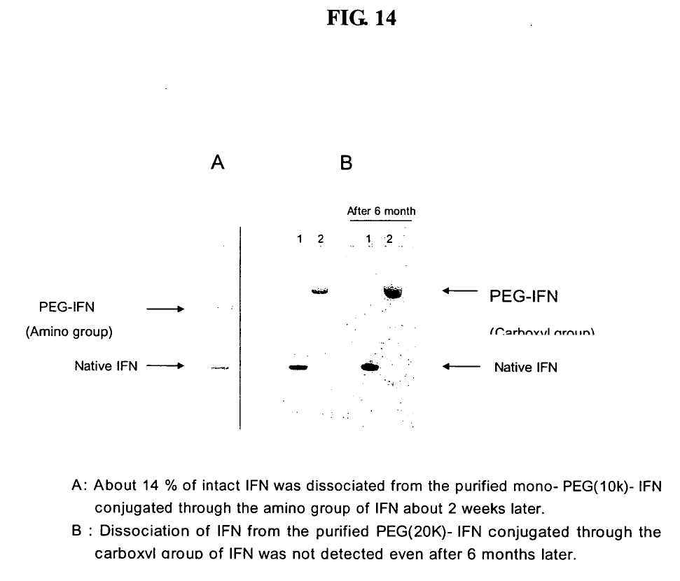

The stability of mPEG(20000)-Hz-IFN prepared and purified in Example 6 and PEG-IFN, wherein branched PEG(10K)2-NHS(attached to the amino group of IFN according to the general method in the literature followed by separating mono PEG-IFN by size exclusion column, Nektar, USA) was determined on SDS-PAGE after incubation at 4° C. in PBS solution by observing the dissociation of intact IFN on the SDS-PAGE. The concentration of each sample was adjusted to the final concentration of 1 mg/ml. As a result, it was observed that about 14% of intact IFN was dissociated from the PEG-IFN conjugated through the amino group of IFN about 2 weeks later, whereas the dissociation of IFN from PEG(20000)-Hz-IFN conjugated through the carboxyl group of IFN of the present invention was not detected even after 6 months later(FIG. 14).

### Example 15

### Preparation of mPEG(5000)-Hz-PTH Conjugate

1 mg of human PTH(parathyroid hormone, 0.00012 mmol, 1-84 aa, Dong-Kook Pharm., Korea) and 3.0 mg of activated mPEG(5000)-Hz(0.0006 mmol, ISU Chemical, Korea) in 0.5 ml of 50 mM MES buffer solution, pH 4.4 were reacted for 10 minutes with stirring at room temperature. 2.5 ul of EDAC(0.00125 mmol, 10-fold molar excess) prepared at a concentration of 100 ug/ul, was then added and reacted for 1 hour with stirring at room temperature. Unreacted mPEG(5000)-Hz and PTH were removed by using Centricon-10(Amicon, USA) and 0.4 mg of mPEG(5000)-Hz-PTH was obtained.

### Example 16

### Preparation of mPEG(12000)-Hz-PTH Conjugates

1 mg of human PTH(0.00012 mmol) and 7.14 mg of activated mPEG(12000)-Hz(0.0006 mmol, 5 fold molar excess, ISU Chemical, Korea) in 0.5 ml of 50 mM MES buffer solution, pH 4.4 were reacted for 10 minutes with stirring at room temperature. 2.5 ul of EDAC(0.00125 mmol, 10-fold molar excess) prepared at a concentration of 100 ug/ul, was then added and reacted for 1 hour with stirring at room temperature. Unreacted mPEG(12000)-Hz and PTH were removed by using Centricon-10(Amicon, USA) and 0.3 mg of mPEG(12000)-Hz-PTH was obtained.

### Example 17

### Preparation of mPEG(20000)-Hz-PTH Conjugates

1 mg of human PTH(0.00012 mmol) and 12 mg of activated mPEG(20000)-Hz(0.0006 mmol, 5 fold molar excess, ISU Chemical, Korea) in 0.5 ml of 50 mM MES buffer solution, pH 4.4 were reacted for 10 minutes with stirring at room temperature. 2.5 ul of EDAC(0.00125 mmol, 10-fold molar excess) prepared at a concentration of 100 ug/ul, was then added and reacted for 1 hour with stirring at room temperature. Unreacted mPEG(20000)-Hz and PTH were removed by using Centricon-30(Amicon, USA) and 0.3 mg of mPEG(20000)-Hz-PTH was obtained.

### Example 18

### Preparation of mPEG(12000)-Hz-PTH Conjugate

1 mg of human PTH(0.00012 mmol) and 14.4 mg of activated mPEG(12000)-Hz(0.0012 mmol, 10 fold molar excess, ISU Chemical, Korea) in 0.5 ml of 50 mM MES buffer solution, pH 2.5 were reacted for 10 minutes with stirring at room temperature. 5 ul of EDAC(0.0025 mmol, 20-fold molar excess) prepared at a concentration of 100 ug/ul, was then added and reacted for 1 hour with stirring at room temperature. Unreacted mPEG(12000)-Hz and PTH were removed by using Centricon-10(Amicon, USA) and 0.2 mg of mPEG(12000)-Hz-PTH was obtained.

### Example 19

### Preparation of mPEG(20000)-Hz-PTH Conjugate

1 mg of human PTH(0.00012 mmol) and 24 mg of activated mPEG(20000)-Hz(0.0012 mmol, 10-fold molar excess, ISU Chemical, Korea) in 0.5 ml of 50 mM MES buffer solution, pH 2.5 were reacted for 10 minutes with stirring at room temperature. 5 ul of EDAC(0.0025 mmol, 20-fold molar excess) prepared at a concentration of 100 ug/ul, was then added and reacted for 1 hour with stirring at room temperature. Unreacted mPEG(20000)-Hz and PTH were removed by using centricon-10(Amicon, USA) and 0.2 mg of mPEG(20000)-Hz-PTH was obtained.

### Example 20

### Analysis of mPEG-Hz-PTH Conjugate PEG-PTH conjugate and native PTH which were obtained from the above examples were determined as following HPLC condition

TABLE 3
Eluting Condition of HPLC
Column: LiChroCART 125-4 RP-8 (5 um)(Merck, USA)
Solvent: A; deionized water containing 0.1% TFA,
B; acetonitrile containing 0.1% TFA
Flow rate: 0.8 ml/min
Detector: UV detector at 220 nm
Injection volume: 20 ul

| number | Time(min) | A (%) | B (%) | Flow rate(ml/min) |
|---|---|---|---|---|
| 1 | 0.00 | 70.0 | 30.0 | 0.800 |
| 2 | 3.00 | 70.0 | 30.0 | 0.800 |
| 3 | 13.00 | 10.0 | 90.0 | 0.800 |
| 4 | 15.00 | 10.0 | 90.0 | 0.800 |
| 5 | 17.00 | 70.0 | 30.0 | 0.800 |
| 6 | 20.00 | 70.0 | 30.0 | 0.800 |

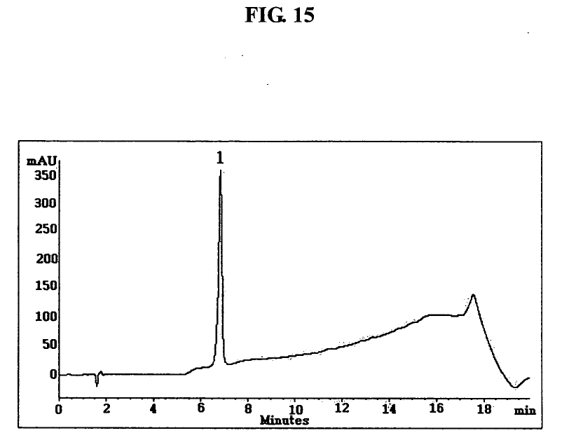

By using LiChroCART 125-4 RP-8 (5 um) for HPLC, only PTH and other proteins were detected at 220 nm whereas no PEG was detected at 220 nm. The RT of PTH was determined by HPLC. A sharp peak of PTH which is not modified by the polymer was seen around 6.8 min, then slowly increased up to about 18 min, and decreased again(FIG. 15).

mPEG-Hz-PTH prepared as described above was eluted at 6.8 min for unreacted PTH and 7.3 min for PEG-PTH, respectively, in the range of the elution conditions between number 1 and 2 in the above table.

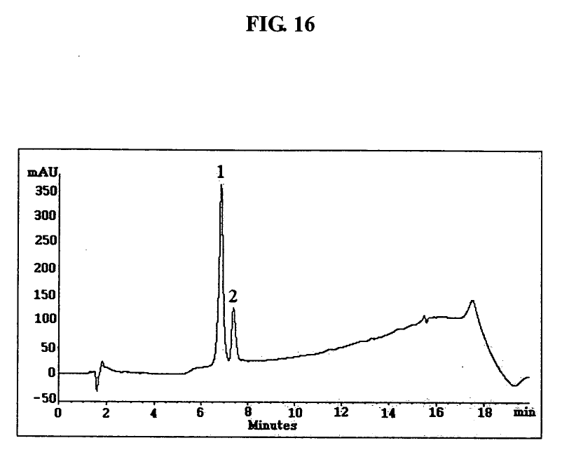

There are three kinds of products(unreacted PTH, mPEG(20000)-Hz-PTH, mPEG(20000)-Hz) present immediately after the conjugation and before purification. Only two peaks, unreacted PTH and mPEG(20000)-Hz-PTH conjugate, were detected at 220 nm on HPLC while unreacted mPEG(20000)-Hz was not detected at 220 nm(FIG. 16).

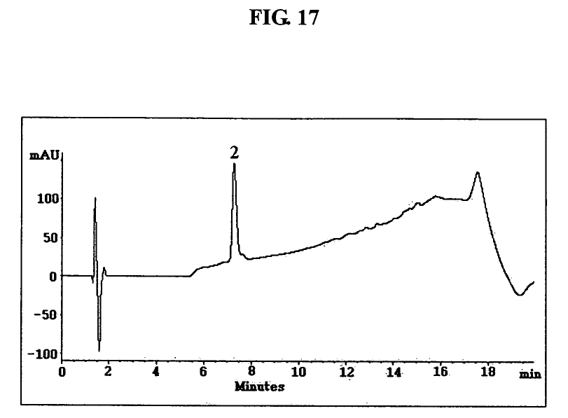

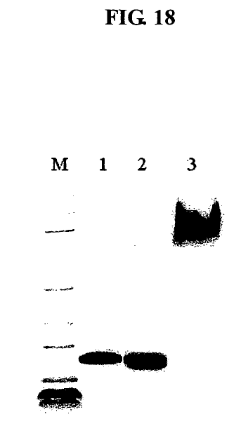

FIG. 17 shows the finally purified mPEG(20000)-Hz-PTH on HPLC and FIG. 18 SDS-PAGE stained with Coomassie blue performed after reacting mPEG(20000)-Hz with PTH.

### Example 21

### Determination of In Vitro Biological Activity of mPEG-PTH Conjugate

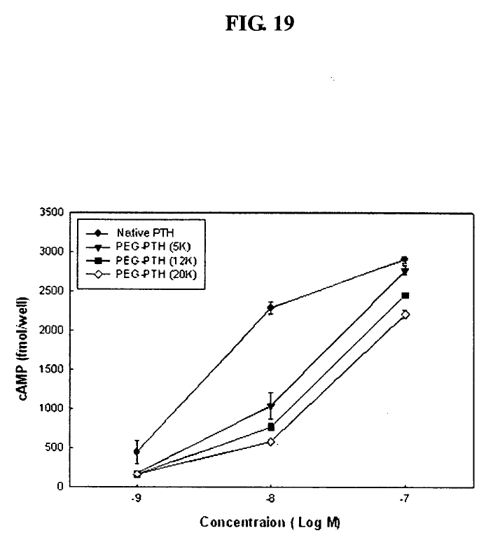

The activated mPEG-Hz having molecular weights of 5000(5K), 12000(12K), and 20000(20K) were used to determine the biological activity according to molecular weight of PEG. The in vitro biological activity of native PTH, mPEG(5000)-Hz-PTH, mPEG(12000)-Hz-PTH, and mPEG(20000)-Hz-PTH was compared by determining the amount of c-AMP synthesized by c-AMP kit(Amersham Pharmacia, RPN 225, USA) using UMR-10⁶ cell line. It was found that the biological activity of mPEG-Hz-PTH was decreased as molecular weight of PEG increased. It was also found that mPEG(5000)-Hz-PTH, mPEG(12000)-Hz-PTH, and mPEG(20000)-Hz-PTH at the concentration of 10⁻⁸ mole retained 40%, 30%, and 20% activity of unreacted PTH, respectively(FIG. 19).

### Example 22

### Determination of Half-Life of mPEG-Hz-PTH

100 ug/kg of PTH and mPEG-Hz-PTH was administered through intravenous injection, respectively to each of male rats weighing 300-350 g. Blood was then withdrawn through the cannula at time intervals of 0, 5, 10, 15, 30, 60, and 120 min after injection. The serum was separated by centrifugation(10,000 rpm, 10 min, 4° C.) and the half life of mPEG-Hz-PTH was indirectly determined by calculating the concentration of remaining PTH by measuring the concentration of cAMP in plasma using c-AMP kit(Amersham Pharmacia, RPN 225, USA).

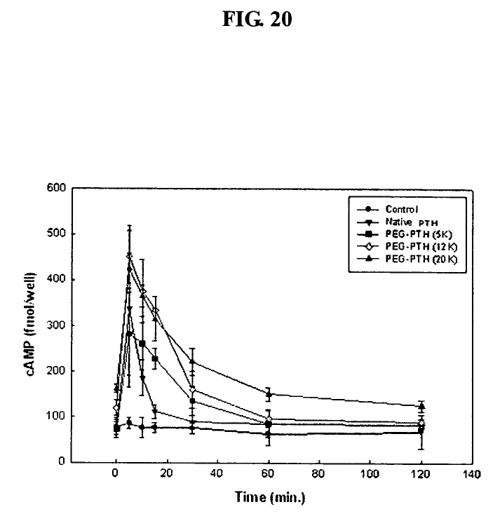

As a result, unreacted PTH and mPEG(5000)-Hz-PTH were not detected after 15 mins after administration. However, mPEG(12000)-Hz-PTH and mPEG(20000)-Hz-PTH were detected after 1 hour and 2 hours, respectively, after administration(FIG. 20).

### Example 23

### Preparation of mPEG(20000)-Hz-EPO Conjugate

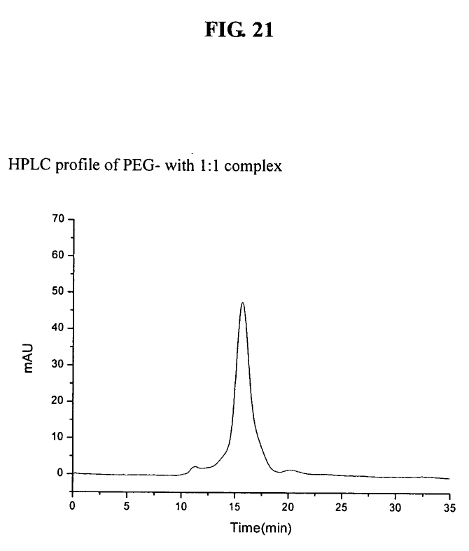

250 ug of EPO solution(0.0000083) was dialyzed(Centricon-10, Amicon, USA) against 50 mM MES buffer solution(pH 3.0) to the final concentration of 5 mg/ml. To this tube, 1.67 mg of mPEG(20000)-Hz(0.000083 mmol, ISU Chemical, Korea) was added, followed by adding 2.4 ul(30-fold molar excess) EDAC solution prepared by dissolving 1 mg of EDAC in 50 ul of d-H₂O. The reaction was carried out for 1 hour at room temperature(20-25° C.) with stirring. After 1 hour, unreacted EPO and excess reagent were removed by size exclusion column or ion-exchange column. The purified PEG-EPO with 1:1 complex was verified by HPLC in FIG. 21.

### Example 24

### Determination of Biological Activity of PEG-EPO Conjugate Cell Proliferation Assay was Performed as Follows

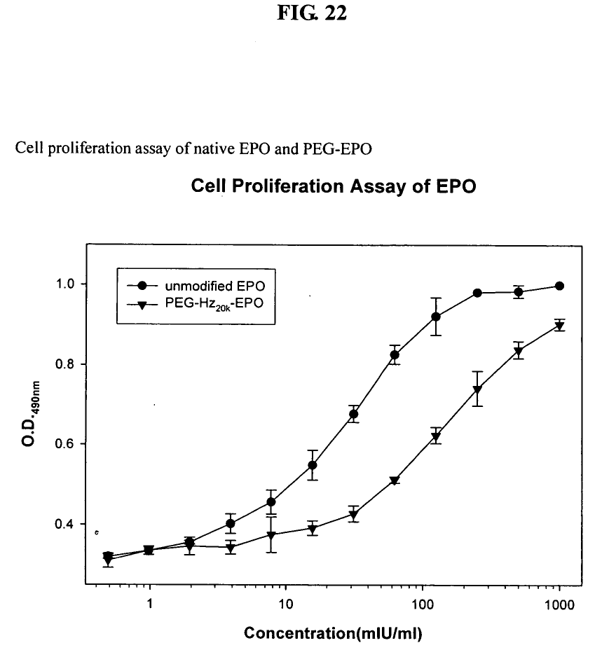

1×10⁶ cells/ml of F-36E were sub-cultured on 60 mm dishes(RPMI-1640 media, 10% FBS, 37° C., 5% CO₂). 80 ul of media (10% RPMI-1640) was added to each well. Each of native EPO(control) and mPEG(20000)-Hz-EPO(Example 23) was diluted to the concentration of 5 mu/μl and added to the 96 well plate followed by serial dilution. 20 ul of 1×10⁶ cells/ml was then added to each well followed by incubation at 37 C for 4 days. Then each well was treated with 50 μl of XTT kit(Roche, Germany) and incubated for another 4 hours at 37° C., and O.D. value of the plate was read at 490 nm using ELISA reader (FIG. 22).

Thus, while there have been described the preferred embodiments of the present invention, those skilled in the art will realize that other embodiments can be made without departing from the spirit of the invention, which includes all such further modifications and changes as come within the meaning, true scope of the claims set forth herein and equivalents thereof. The above examples further describe and demonstrate embodiments within the scope of the present invention. The examples are given solely for the purpose of illustration and are not to be construed as limitations of the present invention, as many variations thereof are possible without departing from the spirit and scope of the invention.

Application of the Present Invention

The present invention provides the biocompatible polymer-biologically active material conjugates in a molar ratio of 1:1 wherein the biocompatible polymer is attached to a carboxyl group of the biologically active material such as proteins or peptides and method of preparation thereof. These conjugates having the increased bioavailability and extended half-life due to their increased in vivo stability can reduce the frequency of administration significantly when used as therapeutic drugs for diseases.

1. A conjugate of biocompatible polymer-biologically active material, wherein the activated biocompatible polymer is conjugated to a carboxyl group of biologically active material at a molar ratio of 1:1.

2. The conjugate according to claim 1, wherein the biocompatible polymer is selected from the group consisting of polyethylene glycol, polypropylene glycol, polyoxyethylene, polytrimethylene glycol, polylactic acid and derivatives thereof, polyacrylic acid and derivatives thereof, poly(amino acid), polyurethane, polyphosphazene, poly(L-lysine), polyalkylene oxide, polysaccharide, dextran, polyvinyl pyrrolidone, polyvinyl alcohol, polyacryl amide, and copolymers thereof.

3. The conjugate according to claim 1, wherein the biologically active material is selected from the group consisting of α-, β-, γ-interferon, parathyroid hormone, asparaginase, arginase, arginine deiminase, adenosine deaminase, superoxide dismutase, endotoxinase, catalase, chymotrypsin, lipase, uricase, adenosine diphosphatase, tyrosinase, glucose oxidase, glucosidase, galactosidase, glucouronidase, hemoglobin, blood factors VII, VIII and IX, immunoglobulin, cytokine, granulocyte colony stimulating factor, granulocyte macrophage colony stimulating factor, platelet derived growth factor, lectin, ricin, tumor necrosis factor, transforming growth factor, epidermal growth factor, calcitonin, insulin, synthetic enkephalin, interleukin, erythropoietin, growth hormone releasing peptide, luteal hormone-releasing hormone and derivatives thereof, hypothalamic releasing factors, calcitonin gene related peptides, thyroid stimulating hormone and thymic humoral factor.

4. The conjugate according to claim 1, wherein the biologically active material is interferon, G-CSF, or parathyroid hormone.

5. A pharmaceutical composition comprising a pharmaceutically acceptable amount of the conjugate according to claim 1 and a pharmaceutically acceptable carrier.

6. A method of preparation of a conjugate of biocompatible polymer-biologically active material, wherein an activated biocompatible polymer is conjugated to a carboxyl group of the biologically active material at a molar ratio of 1:1, comprising the step of conjugating the biologically active material to the activated biocompatible polymer with the stepwise addition of a coupling reagent, wherein the molar ratio of the biologically active material to the activated biocompatible polymer is 1:1 to 1:20, the ratio of the biologically active material to the coupling reagent is 1:1 to 1:50, and pH is in the range of 2 to 5.

7. The method according to claim 6, wherein the biocompatible polymer is activated with a reactive functional group which is able to react with a carboxylic acid and/or a reactive carbonyl group.

8. The method according to claim 6, wherein the biocompatible polymer is selected from the group consisting of polyethylene glycol, polypropylene glycol, polyoxyethylene, polytrimethylene glycol, polylactic acid and derivatives thereof, polyacrylic acid and derivatives thereof, poly(amino acid), polyurethane, polyphosphazene, poly(L-lysine), polyalkylene oxide, polysaccharide, dextran, polyvinyl pyrrolidone, polyvinyl alcohol, polyacryl amide, and copolymers thereof.

9. The method according to claim 6, wherein the biologically active material is selected from the group consisting of α-, β-, γ-interferon, parathyroid hormone, asparaginase, arginase, arginine deiminase, adenosine deaminase, superoxide dismutase, endotoxinase, catalase, chymotrypsin, lipase, uricase, adenosine diphosphatase, tyrosinase, glucose oxidase, glucosidase, galactosidase, glucouronidase, hemoglobin, blood factors VII, VIII and IX, immunoglobulin, cytokine, granulocyte colony stimulating factor, granulocyte macrophage colony stimulating factor, platelet derived growth factor, lectin, ricin, tumor necrosis factor, transforming growth factor, epidermal growth factor, calcitonin, insulin, synthetic enkephalin, interleukin, erythropoietin, growth hormone releasing peptide, luteal hormone-releasing hormone and derivatives thereof, hypothalamic releasing factors, calcitonin gene related peptides, thyroid stimulating hormone and thymic humoral factor.

10. The conjugate of biocompatible polymer-biologically active material prepared according to claim 6, wherein the biocompatible polymer is conjugated to a carboxyl group of the biologically active material at a molar ratio of 1:1.

11. The conjugate of biocompatible polymer-biologically active material, wherein the biocompatible polymer is conjugated to the C-terminus of the biologically active material at a molar ratio of 1:1.

12. The conjugate according to claim 11, wherein the biocompatible polymer is selected from the group consisting of polyethylene glycol, polypropylene glycol, polyoxyethylene, polytrimethylene glycol, polylactic acid and derivatives thereof, polyacrylic acid and derivatives thereof, poly(amino acid), polyurethane, polyphosphazene, poly(L-lysine), polyalkylene oxide, polysaccharide, dextran, polyvinyl pyrrolidone, polyvinyl alcohol, polyacryl amide, and copolymers thereof.

13. The conjugate according to claim 11, wherein the biologically active material is selected from the group consisting of α-, β-, γ-interferon, parathyroid hormone, asparaginase, arginase, arginine deiminase, adenosine deaminase, superoxide dismutase, endotoxinase, catalase, chymotrypsin, lipase, uricase, adenosine diphosphatase, tyrosinase, glucose oxidase, glucosidase, galactosidase, glucouronidase, hemoglobin, blood factors VII, VIII and IX, immunoglobulin, cytokine, granulocyte colony stimulating factor, granulocyte macrophage colony stimulating factor, platelet derived growth factor, lectin, ricin, tumor necrosis factor, transforming growth factor, epidermal growth factor, calcitonin, insulin, synthetic enkephalin, interleukin, erythropoietin, growth hormone releasing peptide, luteal hormone-releasing hormone and derivatives thereof, hypothalamic releasing factors, calcitonin gene related peptides, thyroid stimulating hormone and thymic humoral factor.

14. The conjugate according to claim 13, wherein the biologically active material is interferon, G-CSF, or parathyroid hormone.

15. A pharmaceutical composition comprising a pharmaceutically acceptable amount of the conjugate according to claim 11 and a pharmaceutically acceptable carrier.

16. A method of preparation of a conjugate of biocompatible polymer-biologically active material, wherein an activated biocompatible polymer is conjugated at the C-terminus of the biologically active material with a molar ratio of 1:1, comprising the step of conjugating the biologically active material to the activated biocompatible polymer with stepwise addition of a coupling reagent, wherein the molar ratio of the biologically active material to the activated the biocompatible polymer is 1:1 to 1:20, the ratio of the biologically active material to the coupling reagent is 1:1 to 1:50, and pH is in the range of 2 to 5.

17. The method according to claim 16, wherein the biocompatible polymer is activated with a reactive functional group which is able to react with the a carboxylic acid and/or a reactive carbonyl group.

18. The conjugate according to claim 16, wherein the biocompatible polymer is selected from the group consisting of polyethylene glycol, polypropylene glycol, polyoxyethylene, polytrimethylene glycol, polylactic acid and derivatives thereof, polyacrylic acid and derivatives thereof, poly(amino acid), polyurethane, polyphosphazene, poly(L-lysine), polyalkylene oxide, polysaccharide, dextran, polyvinyl pyrrolidone, polyvinyl alcohol, polyacryl amide, and copolymers thereof.

19. The conjugate according to claim 16, wherein the biologically active material is selected from the group consisting of α-, β-, γ-interferon, parathyroid hormone, asparaginase, arginase, arginine deiminase, adenosine deaminase, superoxide dismutase, endotoxinase, catalase, chymotrypsin, lipase, uricase, adenosine diphosphatase, tyrosinase, glucose oxidase, glucosidase, galactosidase, glucouronidase, hemoglobin, blood factors VII, VIII and IX, immunoglobulin, cytokine, granulocyte colony stimulating factor, granulocyte macrophage colony stimulating factor, platelet derived growth factor, lectin, ricin, tumor necrosis factor, transforming growth factor, epidermal growth factor, calcitonin, insulin, synthetic enkephalin, interleukin, erythropoietin, growth hormone releasing peptide, luteal hormone-releasing hormone and derivatives thereof, hypothalamic releasing factors, calcitonin gene related peptides, thyroid stimulating hormone and thymic humoral factor.

20. The conjugate of biocompatible polymer-biologically active material prepared according to claim 16, wherein the biocompatible polymer is conjugated to the C-terminus of the biologically active material at a molar ratio of 1:1.
